(19) United States
(12) Patent Application Publication
(10) Pub. No.: US 2010/0179639 A1
(43) Pub. Date: Jul. 15, 2010

(54) VASCULAR IMPLANT

(76) Inventors: Stephen Bloor, Alwoodley (GB); Joanne Louise Proffitt, Alwoodley (GB); Paul Armitage, Ackworth (GB); Christine Elizabeth Dawson, Byram (GB)

Correspondence Address: Tyco Healthcare Group LP, 60 MIDDLETOWN AVENUE, NORTH HAVEN, CT, 06473, US

(21) Appl. No.: 12/596,179

(22) PCT Filed: Apr. 15, 2008
(86) PCT No.: PCT/GB2008/001316

§ 371 Date: Mar. 24, 2010

(30) Foreign Application Priority Data
Apr. 16, 2007 (GB) 0707234.1
Apr. 16, 2007 (GB) 0707235.8

(51) Int. Cl. A61F 2/82; B23Q 3/00; C07G 15/00

(52) U.S. Cl. 623/1.15; 269/297; 435/268

(57) ABSTRACT

A vascular implant derived from natural vascular tissue material, wherein the vascular implant is substantially free of non-fibrous tissue proteins, cells and cellular elements and lipids or lipid residues and comprises collagenous material displaying the original fibre architecture and molecular ultrastructure of the natural vascular tissue material from which it is derived.

The present invention relates to a vascular implant, in particular a vascular implant derived from natural vascular tissue.

The tubular blood vessels of the vascular system consist essentially of three concentric layers: the tunica intima, the tunica media and the tunica adventitia. The tunica intima lines the lumen and includes the endothelium. The tunica intima is separated from the tunica media (middle layer) by the internal elastic lamella. The tunica media forms an elastic, muscular tube around the tunica intima, and includes smooth muscle and elastin fibres. In larger arteries, elastin is woven with the collagen fibres. In the aorta, elastin is the dominant protein in the tunica intima. The tunica adventitia is an outer layer of loose connective tissue that connects the vessel to adjacent structures.

Although the basic structure of the vasculature remains constant throughout the circulatory system, there is considerable variation between one type of vessel and another, the major differences being in the density of the smooth muscle cells and the elastin content. Arteries, which have to withstand relatively high blood pressures, are generally thicker and more elastic than veins. The media of a vein has only a little smooth muscle and the adventitia is thick, with an inner layer of dense collagen fibres, then a longitudinally oriented muscular layer interlaced with collagen fibres, and outermost, a layer of collagenous and elastin fibres.

The surgical replacement of diseased or damaged vascular tissue is common. Vascular surgery encompasses surgical treatment, repair and replacement of arteries and veins, as well as treatment for diseases of the peripheral vascular system. Examples of vascular surgery include coronary artery bypass grafts (CABG), arteriovenous access grafts and loops (for dialysis patients), aortic aneurysm repair, and peripheral vascular (PV) repairs, sometimes referred to as below the knee repair. These common surgical procedures may involve the replacement of weakened sections of diseased or damaged vessels, or the introduction of additional vessels in order to bypass damaged areas.

Replacement vessels may be removed from elsewhere within the patient (autograft). Recovering vascular material from the patient for use as a graft has a number of drawbacks and limitations. For example, there is an increase in surgical time required to harvest the autograft, increasing operative risk and adding a further site of surgical trauma. This in turn leads to a longer recovery time due to the morbidity of the donor site and increased amount of postoperative pain. Also, the amount of available viable vascular material may be limited by factors such as the patient's age. For these reasons, vascular graft prostheses are commonly used.

Artificial vascular implants are widely used in vascular surgery. These implants are manufactured from synthetic materials such as PTFE or polyester (Dacron®), which may be woven to provide a smooth, elastic tube that can be cut and manipulated as required. Synthetic vascular devices may be prone to occlusion, particularly when constructed at small diameters. Other drawbacks associated with the use of synthetic vascular implants include poor biocompatibility, increased inflammatory responses, and poor or slow recolonisation of the internal vessel surface following implantation, leading to reduced vascular function and performance. Further complications may include distal embolisation, true and false aneurysms at the site of anastomosis, and erosion into adjacent structures, which can lead to further ailments such as aorta-enteric fistulae.

Vascular grafts derived from isolated biological tissues have also been constructed and used clinically. Vascular implants may be derived from treated human tissue (allograft) or animal sources, in particular bovine tissue. However, a number of potential problems are associated with the use of processed tissue derived from human or certain animal sources as vascular grafts. For example, the use of human tissue is associated with a risk of infection of the recipient of the graft with, for example, hepatitis viruses, HIV or other latent viral or prion disease agents. In addition, human tissue is in short supply.

The use of bovine tissue in the preparation of vascular grafts may lead to allergic reaction and sensitisation in the recipient of the graft, since approximately 2-3% of individuals are known to become sensitised to bovine collagen.

The present invention has been made from a consideration of the aforementioned problems.

According to a first aspect of the present invention there is provided a vascular implant derived from natural vascular tissue material, wherein the vascular implant is substantially free of non-fibrous tissue proteins, cells and cellular elements and lipids or lipid residues and comprises collagenous material displaying the original fibre architecture and molecular ultrastructure of the natural vascular tissue material from which it is derived.

Typically, in addition to collagen the vascular implant also comprises elastin, which plays an important role in providing elasticity to the vessels. Again, the elastin displays the original fibre architecture and molecular ultrastructure of the natural vascular tissue material from which the implant is prepared. The vascular implant may be considered as essentially a matrix of collagen and elastin in which the natural three-dimensional structures of these fibrous tissue proteins are substantially retained. The collagen and elastin content of natural vascular tissues, and the relative amounts of these fibrous tissue proteins, vary considerably between different vessels. The elastin content of larger arteries can be around 50% or more. It will be appreciated that collagen and elastin content of the vascular implant according to the present invention may vary accordingly.

The vascular implant as herein described may be provided as an intact tubular structure, the diameter of which may vary in accordance with the nature of the vascular starting material. By way of illustration, vessels used in the present invention may have a diameter of around 1-20 mm, typically around 5-10 mm, although it will be appreciated that larger or smaller vessels may also usefully be employed. It will also be appreciated that the length of the vascular implant may easily be adjusted by appropriate cutting before or after tissue processing, or at any stage during the processing. Thus, in use, the processed vascular implant may be cut to the correct size for the particular procedure or implant site. Of course, the maximum length of the implant will depend upon the length of the starting material. As a guide, sections of vessel of around 1-50 cm, typically 5-30 cm, more typically 10-20 cm have proved suitable for use in the present invention.

Two or more vascular implants of the present invention may be fastened together, such as in an end-to-end manner. This may be useful where an especially long vascular graft is required. The vascular implant is flexible and is strong enough to hold a suture.

According to a further aspect of the present invention there is provided a process for the manufacture of a vascular implant as herein described, which comprises treating natural vascular tissue material to remove therefrom cells and cellular elements, non-fibrous tissue proteins, lipids and lipid residues, to provide a collagenous material displaying the original fibre architecture and molecular ultrastructure of the natural vascular tissue material from which it is derived.

Whilst any appropriate processing methodology may be used, a particularly suitable process which may be adapted for use in preparing the vascular implant is disclosed in U.S. Pat. No. 5,397,353, the contents of which are incorporated herein by reference. U.S. Pat. No. 5,397,353 describes processing of porcine dermal tissue to provide collagenous implant materials suitable for homo- or hetero-transplantation. The implants retain the natural structure and original architecture of the natural collagenous tissue from which they are derived, so that the molecular ultrastructure of the collagen is retained. The implant materials are non-reactive, any reactive pathological factors having been removed, and provide an essentially inert scaffold of dermal collagen into which host cells infiltrate readily following implantation.

Dermis is a relatively simple structure, in which there is essentially a single layer of interwoven fibres of collagen and some elastin fibres. It has now surprisingly been found that the processing techniques of U.S. Pat. No. 5,397,353 may be applied to more complex vascular tissues, which typically have several different collagen- (and elastin-) containing layers, the three-dimensional structure of which is substantially preserved.

Advantageously, in the vascular implant according to the present invention, the elastin layer of the internal elastic lamella may be substantially preserved, forming an internal, luminal, surface in the processed vascular implant.

Thus, a further aspect of the present invention may be considered a vascular implant derived from natural vascular tissue material, wherein a luminal surface of the vascular implant comprises the internal elastic lamella which displays the original elastin fibre architecture and molecular ultrastructure of the natural vascular tissue material from which it is derived.

According to a further aspect of the present invention there is provided a process for the manufacture of a vascular implant, which comprises treating natural vascular tissue material to remove therefrom cells and cellular elements, non-fibrous tissue proteins, lipids and lipid residues, to provide a vascular implant having a luminal surface comprising internal elastic lamella which displays the original elastin fibre architecture and molecular ultrastructure of the natural vascular tissue material from which it is derived.

The maintenance of the internal elastic lamella is thought to contribute to the prevention of intimal hyperplasia following implantation. Removal or damage to the internal elastic lamella will expose underlying collagen fibres in the vascular implant, which may trigger platelet aggregation and consequent response mechanisms.

The vascular implant according to the present invention performs well following implantation and has good biocompatibility.

Graft thrombosis and intimal hyperplasia are the major factors in graft failure following implantation. In an in vivo model, no thrombus formation or intimal hyperplasia was observed at up to four weeks post-implantation using a vascular implant of the invention. There was little sign of cellular infiltration into the medial layer of the implant or smooth muscle proliferation at up to four weeks. By comparison, an implanted unprocessed vein autograft showed significant intimal hyperplasia and occlusion over the same period.

Advantageously, the vascular implant according to the present invention is readily seeded by host endothelial cells following implantation. Healthy endothelial cells are rapidly laid down on the luminal surface of the implant following implantation, eventually to provide a substantially intact endothelium. Whilst not wishing to be bound by any particular theory, the inventors have hypothesised that intimal hyperplasia may effectively be a ‘race’ between the development of a functional endothelium and smooth muscle proliferation. If smooth muscle proliferation is more rapid, then hyperplasia develops. However, if an intact endothelial layer develops first, this somehow prevents or at least attenuates smooth muscle hyper-proliferation to provide a more normal regenerative profile.

Additional testing of the biocompatibility of the vascular implant of the present invention was carried out by subdermal implantation in a rat model. Porcine-derived vascular implants showed good biocompatibility, with no significant chronic or acute inflammatory response and no other adverse cellular response observed. The collagen and elastin structure of the vascular implant remained intact at up to four weeks. There was evidence of good integration, with host cell infiltration into the adventitial layer.

There was also seeding of endothelial cells on the luminal surface of the vascular implant at the subdermal site. This is a surprising observation, given the lack of vasculature in the subdermal site of implantation or direct blood flow contact of the vascular implant. The vascular implant is treated to remove non-fibrous tissue proteins, such as growth factors. As such, it would be expected that any molecular signals which could influence the recruitment to the internal elastic lamella and/or differentiation of host cells would be stripped from the vascular implant during processing and that exogenous factors such as growth factors would need to be added to the implant in order to re-introduce any signalling capacity. However, it would seem that some signalling functionality remains despite the tissue processing. Although the reasons for these surprising observations are not entirely clear, and without wishing to be bound by any particular theory, it seems possible that the host cells may be responding to ‘signals’ provided by the structure of the collagen, elastin and/or other fibrous tissue proteins of the vascular implant. This could result in recruitment of host cells from the microvasculature and/or differentiation of host cells into endothelial cells. The host cells could be, for example, pre-existing progenitor cells, mesenchymal stem cells, ‘re-programmed’ fibroblasts, or any other cells capable of giving rise to endothelial cells. The signals may be recognised directly by host cells. It is also possible that elements of the vascular implant structure act indirectly on the host cells, perhaps by binding growth factors or signalling molecules in a tissue-specific manner. The signals may reside in a combination of one or more primary, secondary, tertiary or quaternary structural elements of the fibrous tissue proteins of the implant. As such, signalling may be occurring through recognition of a combination of one or more of protein sequences, and one-dimensional topography, two-dimensional topography or three-dimensional topography.

This guided tissue regeneration indicates that the behaviour of cells and tissues in and on the implanted vascular implant is influenced by the matrix structure of the fibrous tissue proteins therein. The matrix exerts a tissue-specific influence, to guide the development of the regenerated tissue, providing for natural, ordered regeneration.

Thus, it is possible that the behaviour of host cells may be influenced and tissue growth guided by tissue-specific elements of the vascular implant, in particular the collagen, elastin and/or other fibrous tissue proteins therein, giving rise to guided tissue regeneration. Clearly, this would be advantageous for the integration of the vascular implant following use in vascular surgery and subsequent regeneration of the vessel at the site of implantation.

A further aspect of the present invention provides a vascular implant as described herein for use in guided tissue regeneration.

An advantage of the vascular implant as described herein is that its performance does not require the addition of exogenous factors, such as growth factors or anti-thrombogenic factors. Thus, whereas in some embodiments it may be desirable to add exogenous factors to the vascular implant, this is not a requirement of the invention. In some embodiments, therefore, the vascular implant may be free from exogenous growth factors and/or anti-thrombogenic factors.

The vascular implant as herein described may also usefully be employed for in vitro regeneration of vascular tissue.

The starting materials for the present invention may be obtained from any human or non-human mammal. In some embodiments, it is preferred that porcine vascular tissue materials are processed to provide the vascular implant, although it will be understood that other mammalian sources may alternatively be employed, such as primates, cows, sheep, horses and goats.

Any vascular tissue may be used as the starting material, including any artery, vein or other, smaller, blood vessel. It has been found that the carotid artery is one particularly suitable source of natural vascular tissue for use in the present invention, owing to its length and relatively few branches. The starting material may comprise porcine carotid artery. It will be appreciated, however, that any vessel of the circulatory system may be employed as a starting material.

The chosen vessel is isolated from the animal and the surrounding connective tissue removed. The vessel may then be cut to the desired size. The outer tissue surrounding the vessel (the tunica adventitia) has been found to play a role in facilitating integration of the vascular implant following implantation. Thus, it is preferred that the tunica adventitia remains intact or at least partially intact. However, during processing of vascular tissue, the tunica adventitia may absorb significant amounts of processing solutions and the resulting increase in weight of the tissue can in some instances cause the vessel to collapse or kink, which may potentially damage the tissue, particularly the internal elastic lamella. To reduce the likelihood of damage, the tunica adventitia may optionally be removed, typically partially removed. The tunica adventitia may be removed by dissection. Typically, about 50% to about 95% by weight of the tunica adventitia may be removed. Around two thirds or more of the tunica adventitia may be removed. For instance, about 80% to about 95% of the tunica adventitia may be removed. This has been found to provide for effective processing, while leaving sufficient tunica adventitia intact to enable good tissue integration. Removal of about 85% to about 90% of the tunica adventitia has been found to give particularly good results. It may be particularly desirable to at least partially remove the tunica adventitia from the end regions of the vessel during processing or use, to facilitate handling and connection of the ends of the implant to the vessels at the site of implantation.

Non-fibrous tissue proteins include glycoproteins, proteoglycans, globular proteins and the like. Cellular elements include antigenic proteins and enzymes and other cellular debris arising from the processing conditions. These portions of the natural tissue material may be removed by treatment with a proteolytic enzyme.

Whilst any proteolytic enzyme which under the conditions of the process will remove non-fibrous tissue proteins can be used, the preferred proteolytic enzyme is trypsin. It has previously been found that above 20° C. the treatment can in some circumstances result in an alteration of the collagen fibre structure leading to a lower physical strength. Moreover, low temperatures discourage the growth of microorganisms in the preparation. It is therefore preferred to carry out the treatment with trypsin at a temperature below 20° C. Moreover, trypsin is more stable below 20° C. and lower amounts of it may be required. Any suitable trypsin concentration may be used, for instance a concentration within the range of around 0.01 g/L to 25 g/L. It has been found that good results can be obtained using 2.5 g/L porcine trypsin, pH 8.

In the context of dermal tissue processing, U.S. Pat. No. 5,397,353 teaches that the tissue should be digested with trypsin over a period of 28 days. However, this has been found to be unsuitable for treatment of vascular tissues, as over-exposure of the vessels to trypsin damages the overall integrity of the implant. Thus, if vessels are treated for 28 days with trypsin, as taught in U.S. Pat. No. 5,397,353, they have a tendency to collapse and lose structural integrity. Digestion with trypsin should therefore typically be carried out for less than 28 days, preferably less than 10 days, more preferably less than 2 days. By way of example, digestion of the vascular tissue with trypsin for around 24 hours has been shown to be suitable, although even shorter digestion times may be suitable for some vessels. Thus, the trypsin digestion may be carried out for around 24 hours or less. It is generally necessary to digest the tissue with trypsin for at least one hour.

It will be appreciated that the reaction conditions for the treatment with trypsin may be routinely adjusted.

One method of removing lipids and lipid residues from the vascular tissue is by the use of a selective enzyme such as lipase. A further, simpler and preferred method is solvent extraction using an organic solvent. Non-limiting examples of suitable solvents include non-aqueous solvents such as acetone, ethanol, ether, or mixtures thereof, acetone being preferred.

Treatment of the vascular tissue with detergents should generally be avoided, as this may damage the natural collagen structure. Thus, embodiments of the process as described herein may exclude treatment with detergents.

It has also been found that treatment with RNase or DNase enzymes can cause damage to the natural collagen structure. Thus, the process as described herein preferably excludes treatment with RNase and/or DNase.

The process may be used to treat vascular tissue material to provide a vascular implant that is substantially free of non-fibrous tissue proteins, cellular elements, and lipids or lipid residues. Those substances said to be “substantially free” of materials generally contain less than 5% of and preferably less than 1% of said materials.

The tissue processing may optionally include a step of treatment with a cross-linking agent. Whilst any cross-linking agent may be used, preferred cross-linking agents include polyisocyanates, in particular diisocyanates which include aliphatic, aromatic and alicyclic diisocyanates as exemplified by 1,6-hexamethylene diisocyanate, toluene diisocyanate, 4,4′-diphenylmethane diisocyanate, and 4,4′-dicyclohexylmethane diisocyanate, respectively. A particularly preferred diisocyanate is hexamethylene diisocyanate (HMDI). Carbodiimide cross-linking agents may also be used, such as 1-ethyl-3-[3-dimethylaminopropyl]carbodiimide hydrochloride (EDC).

The extent to which the vascular-derived material is cross-linked may be varied. Usefully, this may provide a mechanism for controlling the rate of resorption following implantation. The resistance to resorption tends to increase as the extent of cross-linking is increased.

The vascular implant is preferably cross-linked to provide improved resistance to resorption. By way of example, the vascular implant may be cross-linked using HMDI. As a guide, the HMDI may be used at a concentration of around 0.01 g to 0.5 g per 50 g of tissue. If the concentration is too high, this may result in over-cross-linking and foreign body reactions. It has been found that 0.1 g HMDI per 50 g of tissue provides good results. Cross-linking may be carried out for a range of different time periods. By way of example, the tissue may be exposed to the cross-linking agent for between around 1 hour and around 3 days. Typically, cross-linking is carried out for at least 12 hours, preferably at least 20 hours.

It will be appreciated that the cross-linking conditions may routinely be varied in order to adjust the extent of cross-linking.

In one preferred embodiment of the present invention, the vascular tissue is treated with a solvent, preferably acetone, a proteolytic enzyme, preferably trypsin, and a cross-linking agent, preferably HMDI.

Processing of vascular tissue presents certain difficulties with regard to handling of the tissue. In the art, processing of tubular structures is typically carried out by supporting the tubular structure on a mandrel. However, in order to avoid damage to the internal elastic lamella of the vascular implant, the use of a mandrel should preferably be avoided.

The processing of the vascular tissue may be carried out by supporting the vessel at one or both ends, preferably in a generally vertical position. Any means of support may be used. For example, the vessel may be fastened at or towards one or both ends of to a supporting structure outside the vessel (as opposed to a central mandrel). Advantageously, supporting the vessel in this manner ensures that processing solutions are readily able to pass through the lumen of the vessel as well as around the outside, and reduces the risk of the vessel collapsing during processing. This may be achieved, for instance, by hooking or tying of the end(s) of the vessel, for example using wire or similar. A number of separate fastening points may be used. The vessel should preferably be supported in such a way that there is little or no stress or tension exerted on the tissue. The vessel held generally vertically may be exposed to processing solutions pumped upwards through and around the vessel. This helps to ensure that all parts of the vascular tissue are contacted by the processing solutions. The vessel may be placed in a tube or similar container in order to assist passage of the processing solutions. A plurality of tubes may be connected in order to process a plurality of separate implants.

According to a further aspect of the present invention there is provided an apparatus for the processing of vascular tissue according to a process as herein described, the apparatus comprising a tube with an inlet and an outlet, and support means for supporting the vascular tissue in a generally upright position within the tube, wherein the support means does not comprise a mandrel insertable into the lumen of the vascular tissue.

It will be understood that by “generally upright” it is meant that the longitudinal axis of the vessel is generally vertical.

The inlet and outlet are preferably located at or towards opposite ends of the tube.

The support means may comprise a supporting structure which is insertable into the tube. This may comprise, for example, a mesh. The mesh may be made of any suitable material, stainless steel being one example. The vascular tissue may be fastened to the supporting structure at or towards a first end of the vascular tissue, being the upper end when the vascular tissue is in a generally upright position, and optionally at or towards a second, opposite end. Any suitable fastening means may be provided, such as wire or a similar durable, flexible material.

In use, processing solutions may be introduced into the tube via the inlet. The tube is preferably arranged such that the inlet is lower than the outlet. The tube is typically positioned generally vertically. The processing solutions pass out of the tube via the outlet. Having the inlet lower than the outlet, such that the processing solutions flow upwards, helps to minimise air bubbles within the tubes during processing, which may otherwise affect the effectiveness of the processing. Supporting the vessel in a generally upright position reduces the chance of the vessel collapsing during processing, which may lead to damage of the collagen and elastin structure.

A plurality of tubes may be connected in series, the outlet of each tube being connected to the inlet of the next tube. Processing solutions may be pumped through the tubes in series, providing for convenient and efficient processing of a plurality of vascular implant. Alternatively, the tubes may be connected in parallel. Preferably, a separate tube is provided for each sample of vascular tissue to be processed.

The apparatus may further comprise one or more reservoirs for containing the processing solutions. The apparatus may further be provided with a pump for pumping processing solutions. The elements of the apparatus may be connected using any suitable tubing, typically flexible tubing.

A further aspect of the present invention comprises the use of an apparatus as herein described in the manufacture of a vascular implant.

According to a further aspect of the present invention there is provided a vascular implant produced using a process and/or apparatus as herein described.

According to a further aspect of the present invention there is provided a method of treatment comprising the step of surgically implanting into a patient a vascular implant as herein described.

According to a further aspect of the present invention there is provided the use in vascular surgery of a vascular implant as herein described.

According to a further aspect of the present invention there is provided a vascular implant as herein described for use in vascular surgery.

According to a further aspect of the present invention there is provided the use of a vascular implant as herein described for the manufacture of a product for use in vascular surgery.

The skilled person will be aware of the various vascular surgical procedures. Examples may be found in ‘Vascular Surgery: Cases, Questions, and Commentaries’(Geroulakos et al., Springer, 2003). The vascular implant as herein described may be used in any surgical procedure. For example, the vascular implant may be used as a coronary artery bypass graft (CABG), an arteriovenous (AV) access graft or loop, in aortic aneurysm repair, peripheral vascular (PV) repairs, carotid artery repair, and general vascular bypass grafting procedures. This is not an exhaustive list.

Embodiments of the present invention will now be described further in the following non-limiting examples with reference to the accompanying drawings, in which:

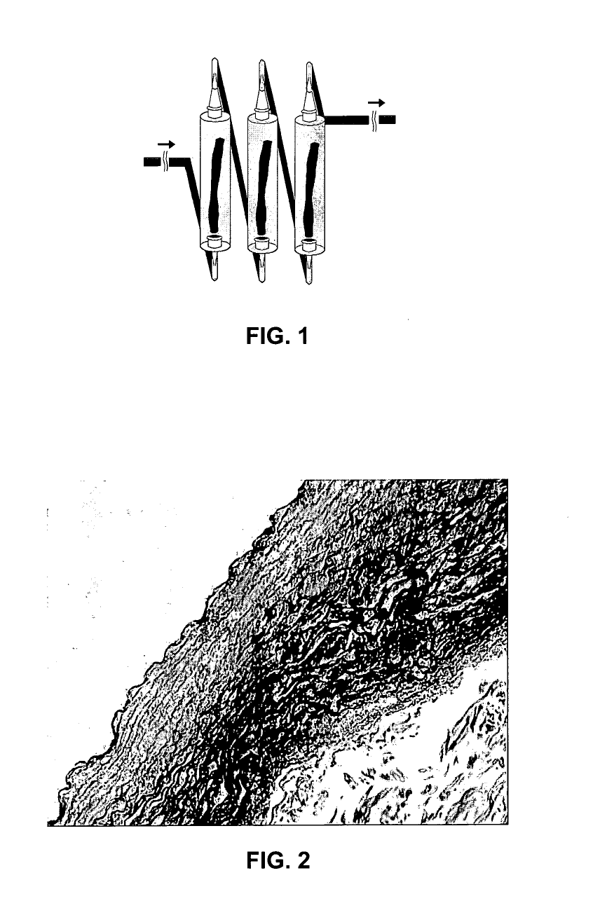

FIG. 1 is a diagrammatic representation of an apparatus according to an embodiment of an aspect of the present invention;

FIG. 2 is a photomicrograph (×200 magnification) of a section of a representative vascular implant according to the present invention, stained with picrosirius red and Millers elastin stain.

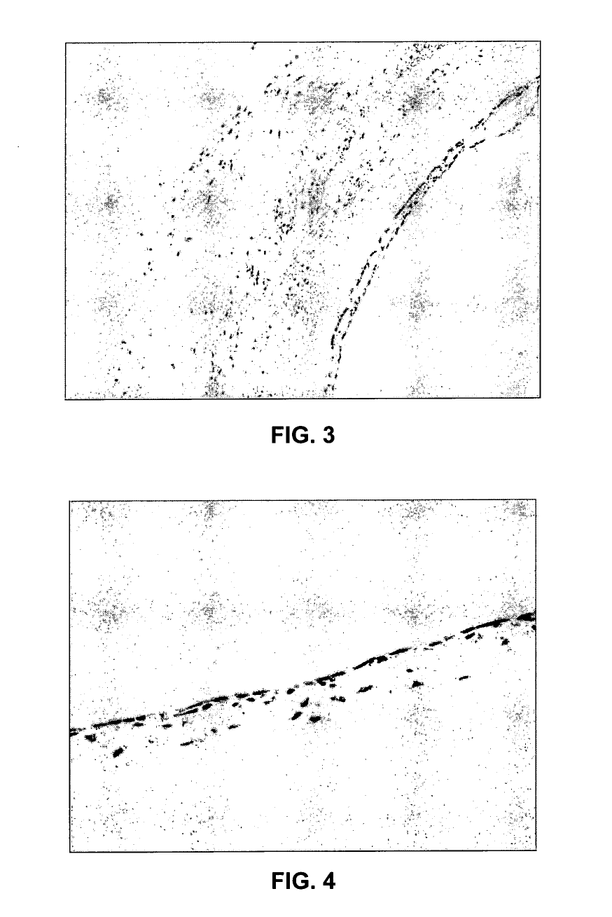

FIG. 3 is a photomicrograph (×200 magnification) of a section of a representative vascular implant according to the present invention 7 days post-implantation in a porcine end-to-end carotid interpositional model, stained with haematoxylin and eosin;

FIG. 4 is a photomicrograph (×400 magnification) of a section of a representative vascular implant according to the present invention 14 days post-implantation in a porcine end-to-end carotid interpositional model, stained with haematoxylin and eosin;

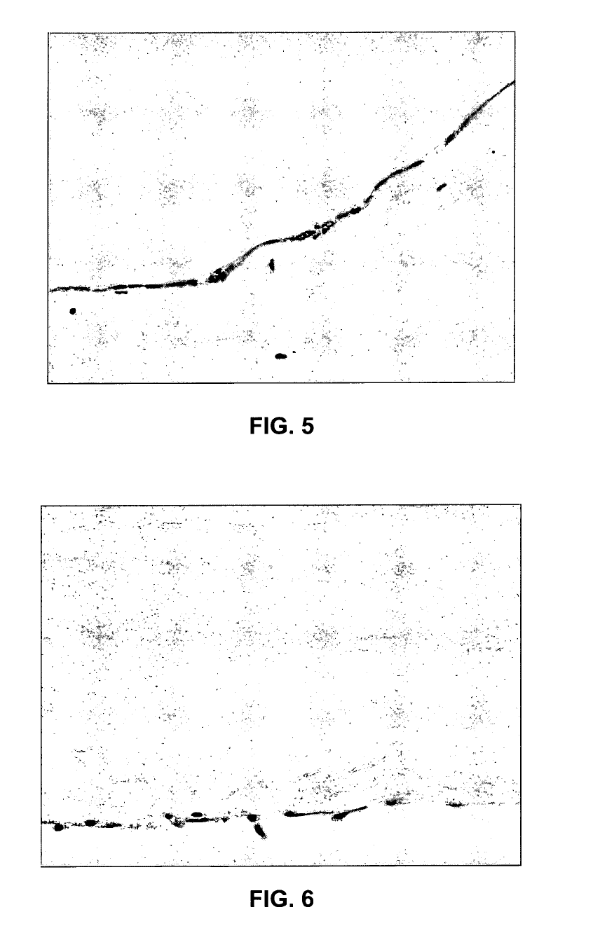

FIG. 5 is a photomicrograph (×400 magnification) of a section of a representative vascular implant according to the present invention 28 days post-implantation in a porcine end-to-end carotid interpositional model, stained with haematoxylin and eosin;

FIG. 6 is a photomicrograph (×400 magnification) of a section of a representative vascular implant according to the present invention 28 days post-implantation subdermally in a rat, stained with haematoxylin and eosin.

### EXAMPLES

### 1. Preparation of Vascular Implant

Sections of porcine carotid arteries (20-30 cm) were harvested from slaughtered sows, immediately placed into 0.9% saline and packed on ice. Following crude dissection to remove most of the connective tissues, some arteries were frozen for prolonged storage and later use. The remaining arteries were processed to provide vascular implants.

Dissection was completed using scissors to cut around two thirds of the tunica adventitia away from the main vessel wall. Each dissected artery was placed individually onto a stainless steel mesh support, secured at each end with stainless steel wire. Each artery/mesh support was placed individually into a glass tube which was then closed at both ends save for an inlet at one end and an outlet at the opposite end. The tubes were positioned generally vertically with the inlet at the bottom and the outlet at the top, and connected in series using flexible tubes, the outlet of each tube being connected to the inlet of the next tube in the series. Once the tubes were connected, processing solutions were pumped through the apparatus using a peristaltic pump to circulate the solutions to and from a main reservoir. The entire apparatus was housed in a temperature-controlled environment. A diagrammatic representation of the processing apparatus is shown in FIG. 1, the arrows indicting the direction of fluid flow.

The vascular tissue was first treated with acetone to remove lipids from the tissue. A 1-hour solvent rinse was followed by a 36-hour solvent rinse. The tissue was then rinsed thoroughly in 0.9% saline to remove the residual acetone from the structure. The material was then placed into trypsin at an activity of 2.5 g/L for 1 day, after which the material was washed with saline to rinse away residual trypsin. After completion of the trypsin digestion, the tissue was rinsed thoroughly in saline. The material was then washed in acetone. There followed a cross-linking step of treatment with HMDI in acetone. A concentration of around 0.1 g HMDI per 50 g of tissue was added. The material was cross-linked for at least 20 hours, rinsed in acetone, and finally rinsed in saline. The vascular implant was gamma-irradiated at 25 kGy.

A sample of the vascular implant was fixed in 10% neutral buffered formal saline. Following fixation, the sample was processed, by routine automated procedures, to wax embedding. 5-micron resin sections were cut and stained using haematoxylin and eosin, picrosirius red and Millers elastin stain.

As shown in FIG. 2, the collagen and (darker-stained) elastin fibre structure is retained in the processed vascular implant. The luminal surface of the vascular implant is formed by the intact internal elastic lamella.

### 2. Functional Implantation of Vascular Implant as Interpositional Graft

Vascular implants prepared as in Example 1 were used in an end-to-end carotid interpositional procedure in Large White/Landrace crossbred female pigs. The animals were pre-treated with an antithrombotic regime of 75 mg aspirin and 75 mg Clopidogrel. The animals were anaesthetised, intubated and ventilated throughout the procedure. Sterile technique was practised. A venous line was placed into a peripheral vein in the ear and glucose saline administered at 800 ml per hour throughout the procedure. A 15-20 cm midline access incision was made from chin to upper sternum. Right and left carotid arteries were exposed and isolated from surrounding tissue. Papaverine and 2% Procaine were administered topically to arteries to ensure vasodilation and 1000 units/kg of heparin were infused into a peripheral ear vein just prior to vessel clamping. The left carotid artery was clamped with single clamps followed by double clamping to provide a length of around 8-10 cm of exposed carotid artery between the clamps. Approximately 6 cm of this artery was resected using a vascular implant of Example 1. The vascular implant was interposed end-to-end into the natural artery and anastomosed with 6/0 or 8/0 continuous sutures. The distal clamps were removed and when the anastomoses stopped oozing the proximal clamps were removed. Pressure was applied until bleeding ceased. The procedure was repeated for the right side. Finally, the access incision was closed with two layers of 2/0 Vicryl® sutures internally and 2/0 Prolene® sutures externally. Ampicillin was administered at 25 mk/kg; Carprofen at 2-4 mg/kg with further doses for 2-3 days; and Ivomec at 0.02 ml/kg. The antithrombotic treatment was continued until harvesting.

After 7, 14 or 28 days, animals were anaesthetised as above and the grafts exposed by careful dissection. The vascular implant was explanted together with the native proximal and distal carotid artery and immediately fixed in 10% neutral buffered formal saline. Following fixation, samples were processed, by routine automated procedures, to wax embedding. 5-micron resin sections were cut and stained using haematoxylin and eosin, picrosirius red and Millers elastin stain.

For comparison, the procedure was also carried out using venous autografts.

In the vein autografts, hyperplasia was observed after 7 days. By 14 days, hyperplasia was well advanced, and after 28 days following implantation hyperplasia was significant, the vessel becoming occluded as a result.

This is in contrast to the results observed using the vascular implant according to the present invention. There was no significant chronic or acute inflammatory response and no other adverse cellular response associated with any of the implants.

The collagen and elastin structure of the vascular implant was maintained 7 days after implantation in the end-to-end carotid interpositional procedure. At the 7-day stage, the external adventitial layer of the implant had begun to integrate with the surrounding tissue, helping to stabilise the graft. There was no cell infiltration into the media of the implant, and no smooth muscle proliferation or presence. Further, there was no evidence of thrombus formation and no platelet adherence to the luminal surface of the implant. Even at this early stage, healthy endothelial cells had begun to seed onto the luminal surface of the graft (see FIG. 3), although not all of the luminal surface was populated with endothelial cells at the 7-day stage.

After 14 days, the collagen and elastin structure of the vascular implant was maintained and the endothelial layer was better developed (see FIG. 4). Seeding of the endothelial layer was not from the ends of the graft, and so the cells would appear to be derived from circulating host endothelial cells and/or progenitor cells. Again, there was no evidence of smooth muscle cell proliferation. FIG. 4 shows that some of the endothelial cells had become characteristically cytoplasmically fused.

By 28 days, the collagen and elastin structure was still intact, including the internal elastic lamella. The endothelial layer was well established and present on almost all of the luminal surface of the graft (see FIG. 5). The endothelial cells appeared healthy and there was extensive cytoplasmic fusion. The adventitia was very well integrated into the host tissue and there were very few cells in the internal media of the implant. There was some evidence of cell proliferation and/or remodelling beneath the endothelial layer. There may have been new tissue, perhaps basement membrane, laid down under the endothelium.

These results demonstrate that the vascular implant of the present invention functioned very well in practice, with no signs of thrombosis or intimal hyperplasia at up to four weeks post-implantation. The vascular implant was readily seeded by host endothelial cells following implantation. It is suggested that the intact internal elastic lamella forming the luminal surface of the implant may be important for achieving good endothelial regeneration. Further, the natural, ordered laying down of the new host endothelium following implantation seemingly results at least in part from the capacity of the implant to induce guided tissue regeneration.

### 3. Subdermal Implantation of Vascular Implant

Vascular implants prepared as in Example 1 were diametrically transected to produce implantable transverse pieces of implant approximately 3 mm in length. Each sample consisted of a full transverse circle of implant. Adult female Sprague Dawley rats were used at 250 g body weight as recipients for these implants. In each animal, two subcutaneous pockets were formed lateral to the midline, one on each side, on the ventral aspect of the animal. For each of these subcutaneous pockets, a single transverse sample of vascular implant was inserted, the pockets closed with a single Vicryl® suture and the midline incision closed with silk suture. At 7 and 28 days post-implantation, samples were explanted together with the surrounding tissue. Samples were fixed immediately in 10% neutral buffered formal saline. Following fixation, all samples were processed, by routine automated procedures, to wax embedding. Two 5-micron sections were cut from each sample; one was stained with haematoxylin and eosin and the other with a combination of picrosirius red and Millers elastin stain.

The collagen and elastin structure of the vessel was well preserved 7 days after subdermal implantation. The implant demonstrated good biocompatibility after 7 days, with no significant chronic or acute inflammatory response and no other adverse cellular response. There was very good integration of the adventitial side of the vascular implant with the local tissue.

It was also found that host endothelial cells were present on the internal lamella of the implant when the samples were evaluated histologically after 7 days. The layer of endothelial cells was even better established after 28 days (see FIG. 6), with some evidence of cytoplasmic fusion. The endothelial cells tested positive for Von Willebrand factor.

The seeding of endothelial cells on the luminal surface of the vascular implant at the subdermal site was a surprising observation, in view of the lack of vasculature in the subdermal site of implantation or direct blood flow contact of the vascular implant. The vascular implant is treated to remove non-fibrous tissue proteins, such as growth factors, and was therefore considered to be essentially inert. However, it would seem that some signalling functionality was retained despite the tissue processing.

The reasons for this surprising result are not entirely clear. It seems possible that the host cells may have responded to ‘signals’ provided by the structure of the collagen, elastin and/or other fibrous tissue proteins of the vascular implant, resulting in recruitment and/or differentiation of host cells. The vascular implant may retain tissue-specific signals in elements of fibrous tissue protein sequence or conformation, which signals are able to influence host cell behaviour within the implant, either directly or indirectly, to give guided tissue regeneration.

The investigation was also repeated using two alternative processes: treatment with RNase and DNase (reference: U.S. Pat. No. 4,776,853, Example 1), and treatment using the Allowash® process (LifeNet; reference: U.S. Pat. No. 6,734,018, Example 1). Samples were explanted after one month and examined as before.

The samples of vascular graft produced using both of these processes induced significant and progressive acute and chronic inflammatory responses in this rodent implantation model. The responses were so severe that, in both cases, there was partial destruction of the integrity of the graft structure and significant host response extending well into the surrounding host tissues.

### 4. Performance Testing of Vascular Implant

Vascular implants prepared as in Example 1 were subjected to a number of mechanical performance tests to confirm suitability for use in surgical procedures.

Burst strength testing was carried out using on methodology described in ANSI/AAMI/ISO 7198:1998/2001/(R) 2004 (cardiovascular implants—tubular vascular prostheses). Suture retention and tensile strength were tested using methodology described in ANSI/AAMI/ISO 7198:1998/2001/(R) 2004 (cardiovascular implants—tubular vascular prostheses), using Hounsfield tensile testing equipment. These mechanical perameters of the vascular implant were found to be within expected physiological ranges for clinical performance.

### 5. Compliance Testing of Vascular Implant

The compliance of vascular implants prepared as in Example 1 was tested and compared to samples of unprocessed human artery and vein and artificial grafts of ePTFE and Dacron®.

A flow circuit was designed to reproduce the haemodynamic characteristics of pulsatile arterial blood flow. This was then used to perfuse segments of the vascular implant of the present invention and the other grafts. The flow model comprised a variable-speed electro-magnetic centrifugal pump (Bio Medicus, Minnetonka, Minn., USA), flexible plastic tubing and a fluid reservoir. A previously governable flow waveform conditioner was sited in series with the circuit and used to generate arterial flow waveforms.

Instantaneous flow rate was measured using a 6 mm tubular flow probe connected to a Transonic medical flowmeter (TMF) system (Transonic Medical System, Ithaca, N.Y., USA). Serial intraluminal pressure measurements were made at discrete sites along the graft using a Millar Mikro-tip catheter transducer (Millar Instruments, Houston, Tex., USA) introduced via a Y-connection port. The perfusion solution was human blood obtained from a blood bank. A gas mixture of 95% oxygen and 5% carbon dioxide was bubbled through the fluid reservoir and the perfusate was maintained at 37° C. via a heat exchanger (Portex, Hythe, UK). Pressure was varied by increasing the level of the fluid reservoir above the graft segment and by varying the diameter of an outflow resistance sited distally to the grafts.

8-10 cm segments of vascular implant (order of 5±1 mm internal diameter), were mounted in series with the flow circuit, exposed to flow and placed in a bath containing normal saline maintained at 37° C. The segments were subject to longitudinal stretch in order to reproduce the likely in vivo conformation. All outputs were fed into a commercial analogue-to-digital data acquisition recording system.

The tests were repeated using segments of human external iliac artery and saphenous vein, retrieved from organ donors. All human vessels were stored in Ringer's lactate solution at 4° C. prior to study use. ePTFE grafts (5 mm ID; Impra, Crawley, UK), uncrimped knitted Dacron® grafts (5 mm ID; Sulzer Vascutek, Inchinnan, UK) were also investigated. Pressure was varied by increasing the level of the blood reservoir above the graft segment and by varying the diameter of an outflow resistance sited distally to the grafts.

Typical flow circuit haemodynamic variables of the perfusion circuit were as follows (mean±SD): mean pressure 30-100 mmHg increased by 10 mmHg increments; pulse pressure 60.2±0.6 mmHg; heart rate 70 bpm; flow rate 155±31 ml/min; perfusion temperature 37±0.5° C.

Mechanical strain and stress were measured using and Instron mechanical tester.

The elastic structure of arteries allows for energy-efficient transmission of pulsatile blood flow together with simultaneous damping of excessive pressure fluctuations. Ideally, any vascular grafts should simulate these biophysical qualities.

Measurement of the elastic behaviour over a range of mean pressures confirmed the anisotropic behaviour of the artery and vein samples. The non-biological prosthetic grafts did not exhibit this behaviour and there was little or no change in the degree of circumferential stretch (compliance) as the mean pressure changed, over the physiological range.

The samples of vascular implant according to the present invention, prepared as in Example 1, showed good compliance and behaved anisotropically. The compliance compared favourably with the results obtained using samples of unprocessed human artery.

It is of course to be understood that the invention is not intended to be restricted by the details of the above specific embodiments, which are provided by way of example only.

1. A vascular implant derived from natural vascular tissue material, wherein the vascular implant is substantially free of non-fibrous tissue proteins, cells and cellular elements and lipids or lipid residues and comprises collagenous material displaying the original fiber architecture and molecular ultrastructure of the natural vascular tissue material from which it is derived.

2. A vascular implant according to claim 1, wherein the implant comprises a portion of elastin displaying the original fiber architecture and molecular ultrastructure of the natural vascular tissue material from which it is derived.

3. A vascular implant according to claim 1, wherein the implant has a tubular structure with a diameter of around 1-20 mm.

4. A vascular implant according to claim 3, wherein the implant has a diameter of around 5-10 mm.

5. A vascular implant according to claim 1, wherein the implant has a length of around 1-50 cm.

6. A vascular implant according to claim 5, wherein the implant has a length of around 5-30 cm.

7. A vascular implant according to claim 6, wherein the implant has a length of around 10-20 cm.

8. A vascular implant according to claim 1, wherein the elastin layer of the internal elastic lamella is substantially preserved, forming an internal, luminal, surface of the vascular implant.

9. A vascular implant according to claim 1, wherein a luminal surface of the vascular implant comprises the internal elastic lamella which displays the original elastin fiber architecture and molecular ultrastructure of the natural vascular tissue material from which it is derived.

10. A vascular implant according to claim 1, wherein the natural vascular tissue material is carotid artery.

11. A vascular implant according to claim 1, wherein the tunica adventitia is at least partially intact.

12. A vascular implant according to claim 11, wherein the tunica adventitia is only partially intact.

13. A vascular implant according to claim 12, wherein about 5% to about 50% by weight of the tunica adventitia is intact.

14. A vascular implant according to claim 13, wherein about 5% to about 20% by weight of the tunica adventitia is intact.

15. A vascular implant according to claim 14, wherein about 10% to about 15% by weight of the tunica adventitia is intact.

16. A vascular implant according to claim 11, wherein the tunica adventitia is at least partially absent from end regions of the implant.

17. A vascular implant according to claim 1 for use in guided tissue regeneration.

18. A process for the manufacture of a vascular implant, comprising treating natural vascular tissue material to remove therefrom cells and cellular elements, non-fibrous tissue proteins, lipids and lipid residues, to provide a collagenous material displaying the original fiber architecture and molecular ultrastructure of the natural vascular tissue material from which it is derived.

19. A process for the manufacture of a vascular implant according to claim 18, wherein the vascular implant has a luminal surface comprising internal elastic lamella which displays the original elastin fiber architecture and molecular ultrastructure of the natural vascular tissue material from which it is derived.

20. A process according to claim 18, wherein the process comprises a step of treatment with a proteolytic enzyme.

21. A process according to claim 20, wherein the proteolytic enzyme is trypsin.

22. A process according to claim 21, wherein treatment with trypsin is carried out for less than 28 days.

23. A process according to claim 22, wherein treatment with trypsin is carried out for less than 10 days.

24. A process according to claim 23, wherein treatment with trypsin is carried out for less than 2 days.

25. A process according to claim 24, wherein treatment with trypsin is carried out for around 24 hours or less.

26. A process according to claim 18, wherein the process comprises a step of removing lipids and lipid residues by solvent extraction using an organic solvent.

27. A process according to claim 26, wherein the solvent is selected from acetone, ethanol, ether, or mixtures thereof.

28. A process according to claim 18, wherein the process comprises a step of treatment with a cross-linking agent.

29. A process according to claim 18, wherein the natural vascular tissue material is supported by means other than a mandrel.

30. A process according to claim 29, wherein the natural vascular tissue material is supported at one or both ends.

31. A process according to claim 30, wherein the natural vascular tissue material is supported in a generally vertical position.

32. A process according to claim 30, wherein the natural vascular tissue material is fastened at or towards one or both ends of to a supporting structure outside the natural vascular tissue material.

33. A process according to claim 31, wherein the natural vascular tissue material is treated with processing solutions pumped upwards through and around the natural vascular tissue material.

34. A process according to claim 18, wherein the process includes a step of removing at least part of the tunica adventitia.

35. A process according to claim 34, wherein about 50% to about 95% by weight of the tunica adventitia is removed.

36. A process according to claim 35, wherein about 80% to about 95% by weight of the tunica adventitia is removed.

37. A process according to claim 36, wherein about 85% to about 90% by weight of the tunica adventitia is removed.

38. A vascular implant produced by a process according to claim 18.

39. An apparatus for the processing of vascular tissue, the apparatus comprising a tube with an inlet and an outlet, and support means for supporting the vascular tissue in a generally upright position within the tube, wherein the support means does not comprise a mandrel insertable into a lumen of the vascular tissue.

40. An apparatus according to claim 39, wherein the inlet and outlet are located at or towards opposite ends of the tube.

41. An apparatus according to claim 39, wherein the support means comprises a supporting structure which is insertable into the tube.

42. An apparatus according to claim 41, wherein the supporting structure comprises a mesh.

43. An apparatus according to claim 39, wherein the tube is arranged such that the inlet is lower than the outlet.

44. An apparatus according to claim 43, wherein the tube is positioned generally vertically.

45. An apparatus according to claim 39, wherein the apparatus comprises a plurality of tubes.

46. An apparatus according to claim 45, wherein the tubes are connected in series, the outlet of each tube being connected to the inlet of the next tube.

47. An apparatus according to claim 45, wherein the tubes are connected in parallel.

48. A vascular implant produced by a process carried out using an apparatus according to claim 39.

49. Use of an apparatus according to claim 39 in the manufacture of a vascular implant.

50. A method of treatment comprising the step of surgically implanting into a patient a vascular implant according to claim 1.

51. Use in vascular surgery of a vascular implant according to claim 17.

52. A vascular implant produced by a process according to claim 18 for use in vascular surgery.

53. Use of a vascular implant produced by a process according to claim 48 for use in vascular surgery.

54. Use of a vascular implant produced by a process according to claim 18 for guided tissue regeneration.
